(19) United States
(12) Patent Application Publication
(10) Pub. No.: US 2019/0076340 A1
(43) Pub. Date: Mar. 14, 2019

(54) COMPOSITIONS AND METHODS FOR PROVIDING FLUORESCENT COSMETICS

(71) Applicant: Imerys USA, Inc., Rosewell, GA (US)

(72) Inventors: Parvin GOLBAYANI, Johns Creek, GA (US); Douglas WICKS, Johns Creek, GA (US)

(21) Appl. No.: 16/085,105

(22) PCT Filed: Mar. 15, 2017
(86) PCT No.: PCT/US17/22495

§ 371 Date: Sep. 14, 2018

(30) Foreign Application Priority Data
Mar. 15, 2016 (EP) 16305281.4

(51) Int. Cl. A61K 8/19; A61K 8/06; A61Q 1/00

(52) U.S. Cl. CPC A61K 8/19; A61K 8/062; A61K 8/064; A61K 2800/10; A61K 2800/5922; A61K 2800/434; A61K 2800/81; A61Q 1/00

(57) ABSTRACT

A cosmetic composition that fluoresces when irradiated with electromagnetic energy is disclosed. The cosmetic composition may comprise a cosmetically accept medium comprising at least one inorganic compound, wherein the inorganic compound is doped with an inorganic fluorescence activator to cause the composition to emit a fluorescent response when irradiated with light, such as UV light. Related products that include such a composition are also disclosed, including a lip stick, an eyeliner, foundation, blush, body paint, a hair gel, or nail polish. There is also a method for imparting a fluorescent response to at least once keratinous surface of a person that comprises applying to the keratinous surface an effective amount of the composition described herein.

### CLAIM FOR PRIORITY

This application claims the benefit of priority from European Patent Application No. 16305281.4, filed Mar. 15, 2016, the contents of which are incorporated by reference.

### DESCRIPTION

### Field

The present disclosure relates to compositions for providing fluorescence to cosmetic formulations. The present disclosure also relates to methods of providing fluorescence to a keratinous surface, such as the skin, nails or hair, by applying the disclosed cosmetic compositions. Finally, the present disclosure relates to cosmetic products comprising the disclosed compositions, such as lip stick, mascara, nail polish, body paint or a makeup product.

### Background

The cosmetic market is a fast growing market characterized by rapid innovation with stiff competition to introduce new value added products. One innovation in cosmetics includes adding organic fluorescent pigments to cosmetic compositions to impart desirable and different optical effects. For example, there are organic fluorescent pigments and dyes used in the cosmetics industry that depend on aromatic azo moieties.

It would be desirable to provide alternative compositions and related methods that providing fluorescence to cosmetic formulations without organic pigments.

### SUMMARY

There is disclosed a cosmetic composition comprising an inorganic compound that fluoresces when irradiated with electromagnetic energy, such as light. According to an aspect, the cosmetic composition may be configured to emit a fluorescent response when irradiated with electromagnetic energy at a predetermined wavelength.

Compositions comprising a fluorescent characteristic as described herein can be used for caring for, or making up the skin of the face or body, or the lips, or keratin fibers, for instance the eyelashes, the eyebrows, or the hair. In an embodiment, the disclosed cosmetic composition comprises a cosmetically acceptable medium comprising at least one inorganic compound, wherein the inorganic compound is doped with an inorganic fluorescence activator to cause the composition to emit a fluorescent response when irradiated with light,

According to some aspects, the fluorescence activator may include at least one mixture, compound, element or alloy of tin, thulium, niobium, cesium, cobalt, manganese, molybdenum, copper, uranium, cesium, thorium, lead, cobalt, iron, strontium, calcium, magnesium, barium, tin, yttrium, thallium, samarium, thulium, cerium, and dysprosium.

The cosmetic composition may be configured for an application that takes advantage of a fluorescent response, and may be in a form of a solution, colloidal dispersion, an oil-in-water emulsion, a water-in-oil emulsion, a suspension, a powder, a cream, a lotion, a gel, a foam, a mousse, or a spray. Non-limiting cosmetic products that may comprise the disclosed cosmetic composition include a lip stick, an eyeliner, foundation, blush, body paint, a hair gel, or nail polish.

There is also a method for imparting a fluorescent response to at least one keratinous surface of a person. In one aspect, the method comprises applying to the keratinous surface an effective amount of a composition comprising: a cosmetically acceptable medium comprising at least one inorganic compound, wherein the inorganic compound is doped with an inorganic fluorescence activator to cause the composition to emit a fluorescent response when irradiated with light

Exemplary objects and advantages will be set forth in part in the description which follows, or may be learned by practice of the exemplary embodiments, It is to be understood that both the foregoing general description and the following detailed description are exemplary and explanatory only and are not restrictive of the invention, as claimed.

### DESCRIPTION OF EXEMPLARY

As used herein, the terms “fluorescence”, “fluoresce”, or “fluorescent response” refer to the emission of electromagnetic energy (e.g., light) by a substance that has absorbed light or other electromagnetic energy or radiation. The emitted light has a longer wavelength, and therefore lower energy, than the absorbed radiation. However, it is meant to generally encompass irradiation at one wavelength and emission of light at a different wavelength, Examples of fluorescence that can be achieved according to the present disclosure include UV to Visible (the material emits visible light after being irradiated with UV light); UV to UV (wherein the emitted and irradiated wavelengths are different); UV to Near IR; and Visible to Near IR.

As used herein the term “UV” or “ultraviolet” light refers to Near UVA (300 nm to 400 nm); UVB (280 nm to 300 nm); and UVA (100 nm to 280 nm).

As used herein, “dopant” refers to a small amount of an impurity that is intentionally added to another material, such as an impurity intentionally added to a carbonate matrix.

As used herein, a “doped” material refers to a material in which a dopant was intentionally introduced during production. For example, a “doped carbonate” refers to a carbonate, such as CaCO₃, gCO₃, BaCO₃, or mixtures thereof, in which a small impurity was intentionally added to change or induce fluorescent response when the carbonate was made.

As used herein, “keratinous” as defined herein refers to human keratinous substances, and may be chosen from, for example, nails, facial skin (including the lips), body skin, and keratinous fibers such as eyelashes, eyebrows, and hair.

The term “cosmetically acceptable medium” denotes a medium that is compatible with application to any keratinous surface of a living being, such as the skin, the lips, or hair of human beings. Consequently, compositions according to the present disclosure may be free of compounds that are incompatible with and/or not tolerated for application to the skin, the lips, and/or the haft,

The term “effective amount” denotes an amount of a composition that emits fluorescent light in the presence of desired irradiation energy, such as ultraviolet light.

Applicants have discovered that fluorescent inorganic fillers, such as precipitated calcium carbonate (PCG), when added to cosmetic preparations, will impart desirable and different optical effects, without the mentioned concerns of organic pigments. According to some embodiments, the disclosed cosmetic composition may comprise a cosmetically acceptable medium; at least one inorganic compound contained in the cosmetically acceptable medium, wherein the inorganic compound is doped with an inorganic fluorescence activator to cause the composition to emit a fluorescent response when irradiated with light.

As described herein, the inorganic compound may include an alkaline earth metal compound, such as an alkaline earth metal carbonate. For example, the alkaline earth metal carbonate may include at least one of calcium carbonate, barium carbonate, and magnesium carbonate. According to some aspects, the alkaline earth metal carbonate may include precipitated calcium carbonate (PCC), magnesium carbonate (PMC), and mixtures thereof, The alkaline earth metal compound may comprise a natural ground calcium and/or magnesium carbonate coated with an alkaline earth metal compound doped with a fluorescence activator.

The inorganic fluorescence activator doped into the inorganic compound typically comprises a mixture, compound, element or alloy tin, thulium, niobium, cesium, cobalt, manganese, molybdenum, copper, uranium, cesium, thorium, lead, cobalt, iron, strontium, calcium, magnesium, barium, tin, yttrium, thallium, samarium, thulium, cerium, and dysprosium. According to some embodiments, the alkaline earth metal compound may have a crystal structure, and the inorganic fluorescence activator may be contained in the crystal structure.

It is possible that the fluorescent response may be configured such that it is not visible to the naked eye, but detectable in the presence of natural light or ultraviolet light. For example, the cosmetic composition may be configured to emit the fluorescent response not when exposed to ambient or natural light, but when irradiated at a predetermined wavelength.

The inorganic fluorescence activator may be configured such that the composition emits radiation at a different wavelength from that with which it was irradiated in the presence of ultraviolet light. According to some embodiments, the composition may be configured to emit energy above, for example, about 350 nanometers in response to radiating the composition with, for example, ultraviolet light below about 450 nanometers. According to some embodiments, the composition may be configured to emit energy above, for example, about 400 nanometers in response to radiating the composition with, for example, ultraviolet light below about 400 nanometers.

According to some aspects, the inorganic fluorescence activator may comprise up to 10 mol % of the alkaline earth metal compound, such as less than 5 mol % of the alkaline earth metal compound, or even less than 1 mol% of the alkaline earth metal compound. According to certain embodiments, these values are noted for inorganic fluorescence activators that comprise CaCO₃ or MgCO₃.

To enhance the fluorescent response, the cosmetic composition may further comprise at least one fluorescent promoter or ligand. Non-limiting examples of the fluorescent promoter or ligand that can be used herein includes fluoride, chloride, bromide, phosphate, sulfate, and combinations thereof.

While these fluorescence activators are generally used in the disclosed composition in carbonate forms, others forms are also possible, such as sulfates (SO₄), phosphates (PO₄³⁻), tungstates (WO₄) and fluorides (F⁻).

According to some aspects, the cosmetic composition disclosed herein may exhibit fluoresce when irradiated with energy having wavelength ranging from about 100 nm to about 1400 nm, such as from about 100 nm to about 750 nm, from about 100 nm to about 400 nm, from about 280 nm to about 1400 nm, from about 315 nm to about 1400 nm, or from about 280 nm to about 750 nm.

The cosmetic composition disclosed herein may comprise mixtures of separately prepared fluorescent carbonates, such as the ones described herein, including mixtures of calcium carbonate, barium carbonate, and magnesium carbonate, to give a multi-wavelength response for a given excitation source. In this embodiment, the intensities of the different wavelength responses can be adjusted by changing the ratios of the separately prepared fluorescent carbonates.

According to still a further aspect, the at least one inorganic fluorescence activator may comprise up to 10 mol % of the inorganic compound, such as less than 5 mol % of the inorganic compound, or even less than 1 mol % of the inorganic compound. According to certain embodiments, these values are noted for inorganic fluorescence activators that comprise CaCO₃ or MgCO₃.

The cosmetic composition according to the present disclosure may additionally comprise at least one standard cosmetic ingredient, which may be chosen from hydrophilic or lipophilic gelling and/or thickening agents, antioxidants, fragrances, preservatives, neutralizing agents, sunscreen agents, vitamins, moisturizing agents, self-tanning compounds, anti-wrinkle active principles, emollients, hydrophilic or lipophilic active principles, agents for combating free radicals, sequestering agents, film-forming agents and mixtures thereof.

The powders can be incorporated into any kind of vehicle that is normally used for cosmetic compositions. For example, the inorganic compounds can be added to solutions, colloidal dispersions, oil-in-water emulsions, water-in-oil emulsions, suspensions, powders, creams, lotions, gels, foams, mousses, sprays and the like.

The emulsions described herein, include oil-in-water (o/w) or water-in-oil (w/o) type dispersion formulations intended for application to the skin, particularly lotions and creams providing the cosmetic properties. The emulsions may contain any of a number of desired “active” ingredients, including the ingredients needed to impart the fluorescent properties, and other ingredients, depending on the intended uses for the compositions.

In one embodiment, the inorganic compounds are used in a powder color cosmetic, such as a face powder, an eye shadow, or a blush. In another embodiment, the powders can be used as part of a liquid cosmetic, such as a liquid foundation, eyeliner, concealer or blush. In addition, the cosmetic composition described herein can be used in solid or semi-solid gel or stick products, such as lipsticks, lip glosses, cream lipsticks, lip or eye pencils, stick foundations, concealers or stick blushes. In one aspect, the disclosed composition may be used in a facial product, such as a foundation, concealer, or blush. The cosmetic composition described herein can further be used in body paints, or hair gels. In another aspect, the cosmetic composition described herein can be used in a nail composition, including but not limited to a nail polish, varnish, gel, or the like.

In one embodiment, there is disclosed a method for imparting a fluorescent response to at least one keratinous surface of a person, such as skin, hair or nails. The disclosed method comprises applying to the keratinous surface an effective amount of the composition comprising; a cosmetically acceptable medium; at least one inorganic compound contained in the cosmetically acceptable medium, wherein the inorganic compound is doped with an inorganic fluorescence activator to cause the composition to emit a fluorescent response when irradiated with light

In one embodiment, the inorganic compound used in the disclosed method comprises an alkaline earth metal compound, such as an alkaline earth metal salt. Non-limiting examples of the alkaline earth metal salt include an alkaline earth metal carbonate selected from calcium carbonate, such as precipitated calcium carbonate, barium carbonate, and magnesium carbonate.

In one embodiment of the disclosed method, the alkaline earth compound comprises a mixture of calcium and magnesium carbonates, such as a mixture of co-precipitated calcium and magnesium carbonates. The mixture of calcium and magnesium carbonates exhibits distinguishable fluorescence properties when exposed to the same excitation source, as result of a difference in the ratios of calcium and magnesium carbonates located in the mixture.

In one embodiment, the method further comprises adding to the composition at least one fluorescent promoter or ligand. Non-limiting examples of fluorescent promoters or ligands that can be used include fluoride, chloride, bromide, phosphate, sulfate, and combinations thereof,

As previously described with the cosmetic composition, the method comprises adding to the inorganic compound an inorganic fluorescence activator. In one embodiment, the activator comprises at least one mixture, compound, element or alloy of tin, thulium, niobium, cesium, and cobalt,

The inorganic fluorescence activator is used in the disclosed method in an amount sufficient to allow the composition to emit fluorescent light in the presence of ultraviolet light, such as up to 10 mol % of the composition, or less than 5 mol % of the composition,

The application of the disclosed cosmetic composition in the method for imparting a fluorescent response to at least one keratinous surface of a person, varies depending on the form of the composition. For example, the cosmetically acceptable medium may be in a variety of different forms including as a solution, a colloidal dispersion, an oil-in-water emulsion, a water-in-oil emulsion, a suspension, a powder, a cream, a lotion, a gel, a foam, a mousse, or a spray.

In addition to the application method being specific for the different forms of the cosmetically acceptable medium, each form may allow for additional fillers to be added to the composition. For example, when the cosmetically acceptable medium is an oil-in-water emulsion, or a water-in-oil emulsion, the composition may further comprise at least one additional filler dispersed in the aqueous phase and in the oily phase.

Non-limiting examples of the at least one additional filler are chosen from waxes, talc, mica, silica, kaolin, polyamide powders, polyethylene powders, starch, hollow microspheres comprise polymers or silica, porous silicas, and ceramic microcapsules.

The method described herein may further comprise adding to the composition at least one cosmetic ingredient chosen from hydrophilic and lipophilic gelling and/or thickening agents, antioxidants, fragrances, preservatives, neutralizing agents, sunscreen agents, vitamins, moisturizing agents, self-tanning compounds, antiwrinkle active principles, emollients, hydrophilic and lipophilic active principles, agents for combating free radicals, sequestering agents, film-forming agents, coloring materials, and mixtures thereof. Coloring materials described herein may be chosen from pigments, pearlescent agents, dyes, and mixtures thereof.

When the inorganic compound disclosed herein comprises an alkaline earth metal compound, such as one doped with an inorganic fluorescence activator, the alkaline earth compound may be obtained via a number of processes. For example, in one embodiment, the composition may comprise precipitated calcium carbonate doped with a fluorescence activator, such as, for example, an impurity such as manganese, Such embodiments may be formed according to the following exemplary reaction:

CaCl₂—MnCl₂ (NH₄)₂CO₃ CaCO₃:Mn.

This exemplary process provides a precipitated calcium carbonate doped with impurities such as manganese, and exhibits a generally rose to orange-red luminescence when irradiated. According to some embodiments of this process, additional inorganic fluorescence activators or impurities may be included, such as, for example, lead, thallium, and cerium salt. Other fluorescence activators are contemplated, and may include at least one mixture, compound, element or ahoy of manganese, molybdenum, copper, uranium, cesium, thorium, lead, cobalt, iron, strontium, calcium, magnesium, barium, tin, yttrium, thallium, samarium, cerium, thulium, and dysprosium.

According to some embodiments, precipitated calcium carbonate may be obtained via another exemplary process. For example, a finely-divided phosphor grade calcium carbonate having a calcite crystalline structure and a very low sodium content may be formed from calcium chloride having a high sodium impurity content, such as, for example, 1.6% sodium chloride. The exemplary process may include forming finely-divided meta-stable vaterite on a continuous basis by continuously adding to an agitated precipitating tank aqueous solutions of calcium chloride and diammonium carbonate in such respective concentrations as to stoichiometrically produce calcium carbonate precipitate and ammonium chloride, The process may further include separating the resulting meta-stable vaterite precipitate from the mother liquor, and then resuspending the separated vaterite in an aqueous medium. Thereafter, the process may include heating the resuspended vaterite to a temperature of at least 80° C. for a sufficient period of time to cause the crystal structure of the vaterite to substantially or completely convert to calcite. The process may also include recovering the resulting calcite, which may have a sodium impurity content in the range of from about 10 parts per million (ppm) to 35 ppm. The impurities such as the fluorescence activators mentioned herein, and others, may be incorporated into the calcite crystalline structure during the reactions and/or following the reactions (e.g., in the form of a coating on the calcite crystalline structure).

According to another exemplary process, precipitated calcium carbonate may be obtained via another process that forms calcium carbonate and ammonium sulfate from gypsum obtained from flue gas desulfurization (FGD), which may be present at electric power plants. According to this exemplary process, FGD gypsum may be obtained from sulfur dioxide SO₂ gas emission control systems used at fossil fuel combustion power plants (e.g., coal-fired power plants) to remove sulfur from the combustion gases using “scrubber” devices, The sulfur dioxide may be derived from any sulfur containing compounds in the fuels. A scrubber uses lime (calcium oxide or calcium hydroxide) or more typically, limestone (calcium carbonate) to react with sulfur dioxide gas to remove the sulfur in a solid form. The scrubbing reaction uses a limestone (CaCO₃)-water slurry to produce calcium sulfite (CaSQ₃) according to the following exemplary reaction:

CaCO₃ (solid)+SO₂ (gas) CaSO₃ (solid)+CO₂ (gas).

Thereafter, the CaSO₃ (calcium sulfite) may be further oxidized to produce CaSO₄.2H₂O (FGD gypsum) according to the following exemplary reaction:

CaSO₃ (solid)+H₂O (liquid)+½ O₂ (gas)→CaSO₄ (solid)+H₂O Hydration CaSO₄½H₂O+1 ½ H₂O→CaSO₄.2H₂O.

Thereafter, the exemplary process may further include a chemical reaction of FGD gypsum (CaSO₄.2H₂O) with ammonium carbonate ((NH₄)₂CO₃) to produce ammonium sulfate ((NH₄)₂SO₄) and calcium carbonate (CaCO₃) according to the following exemplary reaction:

(NH₄)₂CO₃+CaSO₄.2H₂O→(NH₄)₂SO₄₊CaCO₃+2H₂O.

The impurities such as the fluorescence activators mentioned herein, and others, may be incorporated into the resulting precipitated calcium carbonate structure during the reactions and/or following the reactions (e.g., in the form of a coating on the calcium carbonate).

In one embodiment, a traditional process for making PCC (i.e., the lime cycle) may be used. In this process, the fluorescence activator, which may be in the form of a water soluble or water reactive salt, is added to the hydrated lime slurry obtained from the slaking process.

In another embodiment, fluorescent calcium carbonate may be made by mixing solutions of calcium chloride (CaCl₂) and sodium carbonate (Na₂CO₃). The PCC generated (which may comprise mainly vaterite polymorph) may be then dispersed in a solution of carbon quantum dots (CQDs). CQDs may be manufactured from gelatin, as discussed in the examples below, or via processes known in the art. Dispersion of the PCC in the CQD solution may occur, for example, under constant agitation. The final product may emit blue fluorescence under UV (365 nm) irradiation.

In another embodiment, fluorescent calcium carbonate may be made by mixing solutions of calcium sulfate (CaSO₄) and ammonium carbonate ((NH₄)₂CO₃). The PCC generated (which may comprise mainly vaterite polymorph) may be then dispersed in a solution of CQDs. The dispersion of the PCC may occur under constant agitation. The final product may emit blue fluorescence under UV (365 nm) irradiation.

Alternate routes may include using mixtures of vaterite, calcite, and aragonite as substrates, Using amorphous PCC as substrate with subsequent crystallization to vaterite is also possible. Further, ultrafine untreated precipitated calcium carbonates (commercially available as focal® UP) may also be used as a substrate.

The quantum dots may be physisorbed or chemisorbed on salt compounds such as precipitated calcium carbonate and/or ground calcium carbonate.

In another embodiment, a cosmetic may comprise a mineral decorated with a surface-modified precipitated calcium carbonate. Exemplary processes for making a cosmetic comprising a mineral decorated with a surface-modified precipitated calcium carbonate material are also disclosed. These processes may, for example, comprise mixing a calcium source, a mineral, and a carbonate.

The mineral may, for example, comprise one or more of hematite, diatomaceous earth, aluminosilicate, feldspar, palygorskite, nepheline syenite, silica, attapulgite clay, talc, an alkali earth metal carbonate, kaolin, bentonite, calcium carbonate, barium carbonate, and magnesium carbonate. Calcium sources may include, for example, an aqueous calcium solution, for example a solution of one or more of calcium chloride, calcium nitrate, calcium hydroxide, calcium sulfide, and calcium sulfate. Calcium carbonate may, for example, comprise precipitated calcium carbonate, ground calcium carbonate, dolomite, limestone, chalk, and marble. Exemplary carbonates may include NaHCO3, (NH4)2CO3, Na2CO3, Li2CO3, K2CO3, KHCO3, NH4HCO3, and H2CO3.

The mineral may be decorated, for example, when the precipitated calcium carbonate is physisorbed or chemisorbed to at least some fraction of the mineral surface. In one embodiment, the mineral may be encapsulated by the precipitated calcium carbonate. Encapsulated may mean, for example, that the precipitated calcium carbonate covers at least a majority or possibly all of the mineral's surface. For example the encapsulation may comprise a core/shell structure.

The precipitated calcium carbonate may further comprise a metal element. The metal element may comprise one or more of Eu, Y, Sm, La, Ce, Pr, Nd, Pm, Gd, Tb, Dy, Ho,Er, Tm, Yb, Sc, Ti, V, Cr, Mn, Fe, Co, Ni, Cu, Zn, Zr, Nb, Mo, Tc, Ru, Rh, Pd, Ag, Cd, Lu, Hf, Ta, W, Re, Os, Ir, Pt, Au, and Hg. Metal salts thereof may also be included.

The decorated material may fluoresce under UV irradiation, electromagnetic radiation having a wavelength in the range of 200-400 nm, or electromagnetic radiation having a wavelength in the range of 225-300 nm.

Other processes for forming the alkaline earth metal compound doped with an inorganic fluorescence activator are contemplated.

Embodiments consistent with the disclosure may include, for example, the following numbered paragraphs:
1. A cosmetic composition comprising:
a cosmetically acceptable medium comprising at least one inorganic compound, wherein said inorganic compound is doped with an inorganic fluorescence activator to cause the composition to emit a fluorescent response when irradiated.
2. The cosmetic composition of paragraph 1, wherein the inorganic compound comprises an alkaline earth metal compound.
3. The cosmetic composition of paragraph 2, wherein the alkaline earth metal compound comprises an alkaline earth metal salt.
4. The cosmetic composition of paragraph 3, wherein the alkaline earth metal salt comprises at least one alkaline earth metal carbonate selected from calcium carbonate, barium carbonate, and magnesium carbonate.
5. The cosmetic composition of paragraph 4, wherein said calcium carbonate comprises precipitated calcium carbonate.
6. The cosmetic composition of paragraph 4, wherein the alkaline earth compound comprises a mixture of calcium and magnesium carbonates.
7. The cosmetic composition of paragraph 6, wherein said mixture comprises co-precipitated calcium and magnesium carbonates.
8. The cosmetic composition of paragraph 6, wherein the mixture of calcium and magnesium carbonates exhibits distinguishable fluorescence when exposed to the same excitation source.
9. The cosmetic composition of paragraph 8, wherein the distinguishable fluorescent signature comprises a difference in intensity of the emission spectrum resulting from a difference in the ratios of calcium and magnesium carbonates present in the mixture.
10. The cosmetic composition of any preceding paragraph, further providing at least one fluorescent promoter or ligand.
11. The cosmetic composition of paragraph 10, wherein the at least one fluorescent promoter or ligand comprises fluoride, chloride, bromide, phosphate, sulfate, and combinations thereof.
12. The cosmetic composition of any preceding paragraph, wherein the inorganic compound comprises an alkaline earth metal compound having a defined crystal structure, wherein the inorganic fluorescence activator is contained in the crystal structure.
13. The cosmetic composition of any preceding paragraph, wherein the inorganic fluorescence activator comprises at least one mixture, compound, element or alloy of fin, thulium, niobium, cesium, and cobalt.
14. The cosmetic composition of any preceding paragraph, wherein the inorganic fluorescence activator is configured such that the composition emits fluorescent light in the presence of ultraviolet light.
15. The cosmetic composition of any preceding paragraph, wherein the inorganic fluorescence activator comprises up to 10 mol % of the composition.
16. The cosmetic composition of any of paragraphs 1-14, wherein the inorganic fluorescence activator comprises less than 5 mol % of the composition.
17. The cosmetic composition of any preceding paragraph, wherein the cosmetic composition is in the form of a solution, a colloidal dispersion, an oil-in-water emulsion, a water-in-oil emulsion, a suspension, a powder, a cream, a lotion, a gel, a foam, a mousse, or a spray.
18. The cosmetic composition of paragraph 17, wherein the cosmetic composition is in the form of an oil-in-water emulsion, or a water-in-oil emulsion, and the composition further comprises at least one additional filler dispersed in the aqueous phase and in the oily phase.
19. The cosmetic composition of paragraph 18, wherein the at least one additional filler is chosen from waxes, talc, mica, silica, kaolin, polyamide powders, polyethylene powders, starch, hollow microspheres comprising of polymers or silica, porous silicas, and ceramic microcapsules.
20. The cosmetic composition of any preceding paragraph, further comprising at least one coloring material chosen from pigments, pearlescent agents, dyes, and mixtures thereof.
21. The cosmetic composition of any preceding paragraph, further comprising at least one cosmetic ingredient chosen from hydrophilic and lipophilic gelling and/or thickening agents, antioxidants, fragrances, preservatives, neutralizing agents, sunscreen agents, vitamins, moisturizing agents, self-tanning compounds, antiwrinkle active principles, emollients, hydrophilic and lipophilic active principles, agents for combating free radicals, sequestering agents, film-forming agents, and mixtures thereof.
22. A cosmetic product comprising the cosmetic composition of any preceding paragraph.
23. The cosmetic product of paragraph 22, wherein the product is in the form of a lip stick, lip gloss, an eyeliner, foundation, blush, body paint, a hair gel, or nail polish.
24. A method for imparting a fluorescent response to at least one keratinous surface of a person, said method comprising:
applying to said keratinous surface an effective amount of a composition comprising:
a cosmetically acceptable medium comprising at least one inorganic compound, wherein said inorganic compound is doped with an inorganic fluorescence activator to cause the composition to emit a fluorescent response when irradiated.
25. The method of paragraph 24, wherein the inorganic compound comprises an alkaline earth metal compound.
26. The method of paragraph 25, wherein the alkaline earth metal compound comprises an alkaline earth metal salt.
27. The method of paragraph 26, wherein the alkaline earth metal salt comprises at least one alkaline earth metal carbonate selected from calcium carbonate, barium carbonate, and magnesium carbonate.
28. The method of paragraph 27, wherein said calcium carbonate comprises precipitated calcium carbonate.
29. The method of paragraph 27, wherein the alkaline earth compound comprises a mixture of calcium and magnesium carbonates.
30. The method of paragraph 29, wherein said mixture comprises co-precipitated calcium and magnesium carbonates.
31. The method of paragraph 29, wherein the mixture of calcium and magnesium carbonates exhibits distinguishable fluorescent properties when exposed to the same excitation source.
32. The method of paragraph 31, wherein the distinguishable fluorescent properties comprise differences in intensities of the emission spectra resulting from a difference in the ratios of calcium and magnesium carbonates present in the mixture.
33. The method of any of paragraphs 24-32, further comprising adding to the composition at least one fluorescent promoter or ligand,
34. The method of paragraph 33, wherein the at least one fluorescent promoter or ligand comprises fluoride, chloride, bromide, phosphate, sulfate, and combinations thereof.
35. The method of any of paragraphs 24-34, wherein the inorganic compound comprises an alkaline earth metal compound having a defined crystal structure, wherein the inorganic fluorescence activator is contained in the crystal structure.
36. The method of any of paragraphs 24-35, wherein the inorganic fluorescence activator comprises at least one mixture, compound, element or alloy of tin, thulium, niobium, cesium, and cobalt.
37. The method of any of paragraphs 24-36, wherein the inorganic fluorescence activator is configured such that the composition emits fluorescent light in the presence of ultraviolet light.
38. The method of any of paragraphs 24-37, wherein the inorganic fluorescence activator comprises up to 10 mol % of the composition.
39. The method of any of paragraphs 24-37, wherein the inorganic fluorescence activator comprises less than 5 mol % of the composition.
40. The method of any of paragraphs 24-39, wherein the cosmetically acceptable medium is in the form of a solution, a colloidal dispersion, an oil-in-water emulsion, a water-in-oil emulsion, a suspension, a powder, a cream, a lotion, a gel, a foam, a mousse, or a spray.
41. The method of paragraph 40, wherein the cosmetically acceptable medium is an oil-in-water emulsion, or a water-in-oil emulsion, and the composition further comprises at least one additional filler dispersed in the aqueous phase and in the oily phase.
42. The method of paragraph 41, wherein the at least one additional filler is chosen from waxes, talc, mica, silica, kaolin, polyamide powders, polyethylene powders, starch, hollow microspheres comprising of polymers or silica, porous silicas, and ceramic microcapsules.
43. The method of any of paragraphs 24-424, further comprising adding to the composition at least one coloring material chosen from pigments, pearlescent agents, dyes, and mixtures thereof.
44. The method of any of paragraphs 24-33, further comprising adding to the composition at least one cosmetic ingredient chosen from hydrophilic and lipophilic gelling and/or thickening agents, antioxidants, fragrances, preservatives, neutralizing agents, sunscreen agents, vitamins, moisturizing agents, self-tanning compounds, antiwrinkle active principles, emollients, hydrophilic and lipophilic active principles, agents for combating free radicals, sequestering agents, film-forming agents, and mixtures thereof.
45. The method of any of paragraphs 24-44, wherein said keratinous surface comprises the skin, hair or nails.

### EXAMPLE 1

A PCC-carbon quantum dot (CQD) composite was produced using the following steps.

0.8 g gelatin from bovine skin was added to 40 mL water and was dissolved at 40° C. under agitation, Subsequently, the above admixture was poured into a stainless steel autoclave with a teflon liner of 50 mL capacity and heated at 200° C. for 3 hours. Finally, the reactor was automatically cooled to room temperature, The resulting light yellow solution was centrifuged at 16,000 rpm for 30 minutes to remove precipitate and agglomerated particles and then yielded a light brown aqueous solution of CQDs

PCC was generated by mixing solutions of calcium chloride (CaCl₂) and sodium carbonate (Na₂CO₃). The resulting PCC, which comprised mainly the vaterite polymorph, was dispersed in the CDQs solution under constant agitation for 2 hours. The resulting product emitted blue fluorescence under UV (365 nm) irradiation. The resulting fluorescent material may be added to a cosmetic composition, such as, for example, lip stick, mascara, nail polish, body paint or a makeup product.

As an alternate route, PCC was generated by mixing solutions of calcium sulfate (CaSO₄) and ammonium carbonate (NH₄)₂CO₃). The resulting PCC, which comprised mainly the vaterite polymorph, was dispersed in the CDQs solution under constant agitation for 2 hours. The resulting product emitted blue fluorescence under UV (365 nm) irradiation, The resulting fluorescent material may be added to a cosmetic composition, such as, for example, lip stick, mascara, nail polish, body paint or a makeup product.

Alternate routes include using mixtures of vaterite, calcite, and aragonite as substrates. Using amorphous PCC as substrate with subsequent crystallization to vaterite is also possible. Further, ultrafine untreated precipitated calcium carbonates (commercially available as Socal® UP) could also be used as a substrate.

### EXAMPLE 2

Diatomaceous earth was decorated with precipitated calcium carbonate using the following method:

First, 0.036 mol CaCl₂ in 250 mL of water was dissolved with 0.01 mol percent Eu as EuCl₂. A sample of 3.66 g diatomaceous earth was added to the mixture. After that, 0.073 mol NaHCO₃ was also added. The resulting mixture was mixed, filtered, washed, and dried 120° C. for 3 hrs. The resulting product emitted red/orange fluorescence under UV (254 nm) irradiation. The resulting fluorescent material may be added to a cosmetic composition, such as, for example, lip stick, mascara, nail polish, body paint or a makeup product.

Other embodiments of the invention will be apparent to those skilled in the art from consideration of the specification and practice of the exemplary embodiments disclosed herein. It is intended that the specification and examples be considered as exemplary only, with a true scope and spirit of the invention being indicated by the following claims.

1. A cosmetic composition comprising:
a cosmetically acceptable medium comprising at least one inorganic compound, wherein said inorganic compound is doped with an inorganic fluorescence activator to cause the composition to emit a fluorescent response when irradiated.

2. The cosmetic composition of claim 1, wherein the inorganic compound comprises an alkaline earth metal compound or an alkaline earth metal salt.

3. (canceled)

4. The cosmetic composition of claim 3, wherein the alkaline earth metal salt comprises at least one alkaline earth metal carbonate selected from calcium carbonate, barium carbonate, and magnesium carbonate.

5. (canceled)

6. The cosmetic composition of claim 4, wherein the alkaline earth compound comprises a mixture of calcium and magnesium carbonates.

7. The cosmetic composition of claim 6, wherein said mixture comprises co-precipitated calcium and magnesium carbonates.

8. The cosmetic composition of claim 6, wherein the mixture of calcium and magnesium carbonates exhibits distinguishable fluorescence when exposed to the same excitation source.

9. The cosmetic composition of claim 8, wherein the distinguishable fluorescent signature comprises a difference in intensity of the emission spectrum resulting from a difference in the ratios of calcium and magnesium carbonates present in the mixture.

10. The cosmetic composition of claim 1, further providing at least one fluorescent promoter or ligand.

11. The cosmetic composition of claim 10, wherein the at least one fluorescent promoter or ligand comprises fluoride, chloride, bromide, phosphate, sulfate, and combinations thereof.

12. The cosmetic composition of claim 1, wherein the inorganic compound comprises an alkaline earth metal compound having a defined crystal structure, wherein the inorganic fluorescence activator is contained in the crystal structure.

13. The cosmetic composition of claim 1, wherein the inorganic fluorescence activator comprises at least one mixture, compound, element or alloy of tin, thulium, niobium, cesium, and cobalt.

14. The cosmetic composition of claim 1, wherein the inorganic fluorescence activator is configured such that the composition emits fluorescent light in the presence of ultraviolet light.

15. The cosmetic composition of claim 1, wherein the inorganic fluorescence activator comprises up to 10 mol % of the composition.

16. The cosmetic composition of claim 1, wherein the inorganic fluorescence activator comprises less than 5 mol % of the composition.

17. The cosmetic composition of claim 1, wherein the cosmetic composition is in the form of a solution, a colloidal dispersion, an oil-in-water emulsion, a water-in-oil emulsion, a suspension, a powder, a cream, a lotion, a gel, a foam, a mousse, or a spray.

18. The cosmetic composition of claim 17, wherein the cosmetic composition is in the form of an oil-in-water emulsion, or a water-in-oil emulsion, and the composition further comprises at least one additional filler dispersed in the aqueous phase and in the oily phase.

19. The cosmetic composition of claim 18, wherein the at least one additional filler is chosen from waxes, talc, mica, silica, kaolin, polyamide powders, polyethylene powders, starch, hollow microspheres comprising of polymers or silica, porous silicas, and ceramic microcapsules,

20. The cosmetic composition of claim 1, further comprising at least one coloring material chosen from pigments, pearlescent agents, dyes, and mixtures thereof

21. The cosmetic composition of claim 1, further comprising at least one cosmetic ingredient chosen from hydrophilic and lipophilic gelling and/or thickening agents, antioxidants, fragrances, preservatives, neutralizing agents, sunscreen agents, vitamins, moisturizing agents, self-tanning compounds, antiwrinkle active principles, emollients, hydrophilic and lipophilic active principles, agents for combating free radicals, sequestering agents, film-forming agents, and mixtures thereof

22. A cosmetic product comprising the cosmetic composition of claim 1, wherein the product is in the forms of a lip stick, lip gloss, an eyeliner, foundation, blush, bodypaint, a hair gel, or nail polish.

23-45. (canceled)
